(19) United States
(12) Patent Application Publication
(10) Pub. No.: US 2019/0290924 A1
(43) Pub. Date: Sep. 26, 2019

(54) IMPLANTABLE PROSTHESES FOR REDUCING VISIBILITY OF BULGING FROM IMPLANTED MEDICAL DEVICES

(71) Applicant: Boston Scientific Neuromodulation Corporation, Valencia, CA (US)

(72) Inventor: Jeffery Van Funderburk, Stevenson Ranch, CA (US)

(21) Appl. No.: 16/358,174

(22) Filed: Mar. 19, 2019

Related U.S. Application Data
(60) Provisional application No. 62/647,105, filed on Mar. 23, 2018.

(51) Int. Cl. A61N 1/375; A61N 1/05; A61N 1/36; A61N 1/378; A61N 1/372

(52) U.S. Cl. CPC A61N 1/37514; A61N 1/0539; A61N 1/0534; A61N 1/3787; A61N 1/37223; A61N 1/3605

(57) ABSTRACT

A cranial prosthesis for implanting over a portion of a patient's skull includes a body having a first major surface configured for positioning against the patient's skull; a second major surface opposite to the first major surface and forming a contour along which the patient's scalp is disposed against; and an outer perimeter defining a boundary between the first and second major surfaces. A cavity is defined along a portion of the first major surface and is configured for receiving and covering a portion of a medical device extending outwardly from the patient's skull. A tapered region extends radially outward along the body toward the outer perimeter and tapers the contour of the second major surface to reduce visibility of bulging along the patient's scalp caused by the portion of a medical device extending outwardly from the patient's skull when the prosthesis is disposed over the implanted medical device.

### CROSS-REFERENCE TO RELATED APPLICATIONS

This application claims the benefit under 35 U.S.C. § 119(e) of U.S. Provisional Patent Application Ser. No. 62/647,105, filed Mar. 23, 2018, which is incorporated herein by reference.

### FIELD

The present disclosure is directed to the area of implantable medical devices and making and using such devices. The present disclosure is also directed to prostheses for reducing visibility of bulging from implanted medical devices, as well as methods of making and using the prostheses and implantable medical devices.

### BACKGROUND

Implantable medical devices, such as electrical stimulation systems, have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator) and one or more stimulator electrodes. The one or more stimulator electrodes can be disposed along one or more leads, or along the control module, or both. The stimulator electrodes are in contact with or near the nerves, muscles, or other tissue to be stimulated. The pulse generator in the control module generates electrical pulses that are delivered by the electrodes to body tissue.

### BRIEF SUMMARY

In some aspects, a cranial prosthesis for implanting over a portion of a patient's skull includes a body having a first major surface configured for positioning against the patient's skull upon implantation of the cranial prosthesis; a second major surface opposite to the first major surface and forming a contour along which the patient's scalp is disposed against upon implantation of the cranial prosthesis; and an outer perimeter defining a boundary between the first and second major surfaces. A cavity is defined along a portion of the first major surface and is configured for receiving and covering a portion of a medical device extending outwardly from the patient's skull. A tapered region extends radially outward along the body toward the outer perimeter. The tapered region tapers the contour of the second major surface to reduce visibility of bulging along the patient's scalp caused by the portion of a medical device extending outwardly from the patient's skull when the prosthesis is disposed over the implanted medical device.

In some aspects, the tapered region extends along no less than 50% of the outer perimeter of the body. In some aspects, the tapered region extends radially outward along the body from an inner boundary toward the outer perimeter, and the inner boundary is positioned radially inward from the outer perimeter by a distance of no less than 3 cm. In some aspects, the tapered region extends to the outer perimeter of the body.

In some aspects, the body defines at least one attachment aperture configured for receiving a fastener for fastening the cranial prosthesis to the skull. In some aspects, the first major surface defines a channel extending between the cavity and the outer perimeter, the channel configured to receive a portion of an electrical stimulation lead extending along the skull. In some aspects, the body defines one or more surface features configured for facilitating tissue ingrowth.

In some aspects, the second major surface is convex along at least two nonparallel axes. In some aspects, the first major surface is concave along at least two nonparallel axes. In some aspects, the body includes a height defining a smallest distance to the second major surface from a given location along the first major surface, and the height of the body gets smaller along the tapered region moving towards the outer perimeter.

In other aspects, a method of implanting a medical device within a patient includes positioning the medical device on or in the patient's skull with a portion of the medical device extending outwardly from the skull. Any of the above-described cranial prostheses defining a cavity are disposed over the skull with the portion of the medical device extending outwardly from the skull disposed in the cavity of the cranial prosthesis. The skull and cranial prosthesis are covered with the patient's scalp. The contour of the second major surface along the tapered region of the cranial prosthesis reduces visibility through the scalp of bulging caused by the portion of the medical device extending outwardly from the skull.

In some aspects, positioning the medical device on or in the patient's skull with a portion of the medical device extending outwardly from the skull includes forming a burr hole in the patient's skull and disposing a burr hole plug in the burr hole, the burr hole plug at least partially extending outwardly from the skull.

In some aspects, positioning the medical device on or in the patient's skull with a portion of the medical device extending outwardly from the skull includes mounting a pulse generator to the patient's skull with at least a portion of the pulse generator extending outwardly from the skull.

In other aspects, an implantable medical device kit includes an electrical stimulation system and any of the above-described cranial prostheses defining a cavity. The burr hole plug is configured to dispose in a burr hole formed in a skull of a patient. The burr hole plug includes a cover assembly, an electrical stimulation lead, and a pulse generator. The burr hole plug is disposed over the burr hole and at least partially extends outwardly from an outer surface of the patient skull. The electrical stimulation lead is configured to extend through the burr hole cover and into the burr hole from a location at least partially external to the patient's skull. The pulse generator is configured to generate stimulation energy and is coupleable with the electrical stimulation lead. The pulse generator is configured for implanting into the patient at a location at least partially external to the patient's skull. The cranial prosthesis is configured to reduce visibility of bulging along the scalp caused by the portion of the electrical stimulation system extending outwardly from the patient's skull when the cranial prosthesis is disposed over a portion of the electrical stimulation system.

In some aspects, the cranial prosthesis is configured to reduce visibility of bulging along the scalp caused by the burr hole plug when the cranial prosthesis is disposed over the burr hole plug. In some aspects, the pulse generator is mounted on the patient's skull and extends outwardly from the outer surface of the patient skull, and the cranial prosthesis is configured to reduce visibility of bulging along the scalp caused by the pulse generator when the cranial prosthesis is disposed over the pulse generator.

In yet other aspects, a cranial prosthesis for implanting over a portion of a patient's skull includes a body having a first major surface configured for positioning against the patient's skull upon implantation of the cranial prosthesis; a second major surface opposite to the first major surface and forming a contour along which the patient's scalp is disposed against upon implantation of the cranial prosthesis; and an outer perimeter defining a boundary between the first and second major surfaces. An inner wall forming a central opening extends between the first and second surfaces. The inner wall is configured for receiving a portion of an implanted medical device extending outwardly from the patient's skull. A tapered region extends radially outward along the body toward the outer perimeter. The tapered region tapers the contour of the second major surface to reduce visibility of bulging along the patient's scalp caused by the portion of a medical device extending outwardly from the patient's skull when the prosthesis is disposed over the implanted medical device.

In some aspects, the body includes a height defining a smallest distance to the second major surface from a given location along the first major surface, where the height of the body along the inner wall is no greater than a distance that the portion of the implanted medical device extending outwardly from the patient's skull extends outwardly from the patient's skull.

In still yet other aspects, a method of implanting a medical device within a patient includes positioning the medical device on or in the patient's skull with a portion of the medical device extending outwardly from the skull. Any of the above-described cranial prostheses defining a central opening are disposed over the skull with the portion of the medical device extending outwardly from the skull disposed in the central opening of the cranial prosthesis. The skull and cranial prosthesis are covered with the patient's scalp. The contour of the second major surface along the tapered region of the cranial prosthesis reduces visibility through the scalp of bulging caused by the portion of the medical device extending outwardly from the skull.

In other aspects, an implantable medical device kit includes an electrical stimulation system and any of the above-described cranial prostheses defining a central opening. The burr hole plug is configured to dispose in a burr hole formed in a skull of a patient. The burr hole plug includes a cover assembly, an electrical stimulation lead, and a pulse generator. The burr hole plug is disposed over the burr hole and at least partially extends outwardly from an outer surface of the patient skull. The electrical stimulation lead is configured to extend through the burr hole cover and into the burr hole from a location at least partially external to the patient's skull. The pulse generator is configured to generate stimulation energy and is coupleable with the electrical stimulation lead. The pulse generator is configured for implanting into the patient at a location at least partially external to the patient's skull. The cranial prosthesis is configured to reduce visibility of bulging along the scalp caused by the portion of the electrical stimulation system extending outwardly from the patient's skull when the cranial prosthesis is disposed over a portion of the electrical stimulation system.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system;

FIG. 2 is a schematic side view of one embodiment of an electrical stimulation lead;

FIG. 3 is a schematic overview of one embodiment of components of a stimulation system, including an electronic subassembly disposed within a control module;

FIG. 4A is a schematic top view of one embodiment of a low-profile control module disposed along an outer surface of a skull and two leads extending from the control module and into the skull via burr holes covered with burr-hole covers;

FIG. 4B is a schematic front view of one embodiment of the low-profile control module of FIG. 4A disposed along an outer surface of a skull and two leads extending from the control module and into the skull via burr holes covered with burr-hole covers;

FIG. 5A is a schematic top view of another embodiment of a low-profile control module disposed along an outer surface of a skull and two leads extending from the control module and into the skull via burr holes covered with burr-hole covers;

FIG. 5B is a schematic front view of one embodiment of the low-profile control module of FIG. 5A disposed along an outer surface of a skull and two leads extending from the control module and into the skull via burr holes covered with burr-hole covers;

FIG. 6 is a schematic cross-sectional view of one embodiment of a burr hole plug disposed in a burr hole formed in a patient's skull, the burr hole plug extending outwardly from the skull and forming a bulge visible through the patient's scalp;

FIG. 7A is a schematic cross-sectional view of one embodiment of a prosthesis disposed over the patient's skull and burr hole plug of FIG. 6, the prosthesis including a tapered region extending outwardly from the burr hole plug along the skull;

FIG. 7B is a schematic cross-sectional view of one embodiment of the patient's scalp laid over the prosthesis and skull of FIG. 7A, the tapered region of the prosthesis smoothing out the bulge of FIG. 6 to reduce visibility of the bulge;

FIG. 8A is a schematic side view of one embodiment of the prosthesis of FIGS. 7A-7B;

FIG. 8B is a schematic cross-sectional view of one embodiment of the prosthesis of FIGS. 7A-7B;

FIG. 8C is a schematic bottom view of one embodiment of the prosthesis of FIGS. 7A-7B;

FIG. 9 is a schematic bottom view of one embodiment of a prosthesis suitable for disposing over an implanted medical device protruding from a patient's skull, the prosthesis having an oval-shaped body;

FIG. 10 is a schematic bottom view of one embodiment of a prosthesis suitable for disposing over an implanted medical device protruding from a patient's skull, the prosthesis having a capsule-shaped body;

FIG. 11 is a schematic bottom view of one embodiment of a prosthesis suitable for disposing over an implanted medical device protruding from a patient's skull, the prosthesis defining a cavity for receiving at least a portion of the implanted medical device that is off-center along the body of the prosthesis;

FIG. 12 is a schematic bottom view of one embodiment of a prosthesis suitable for disposing over one or more implanted medical devices protruding from a patient's skull at multiple locations, the prosthesis defining two cavities for receiving multiple devices or portions of devices, the prosthesis also defining channels for receiving and routing additional medical devices or portions of medical devices;

FIG. 13 is a schematic bottom view of one embodiment of a prosthesis suitable for disposing over one or more implanted medical devices protruding from a patient's skull at multiple locations, the cavities including a cavity having a non-circular shape;

FIG. 14A is a schematic bottom view of one embodiment of a prosthesis suitable for disposing over one or more implanted medical devices protruding from a patient's skull at multiple locations, the prosthesis including a perforated line for facilitating cutting or tearing away a portion of the prosthesis;

FIG. 14B is a schematic bottom view of one embodiment of the prosthesis of FIG. 14A after cutting or tearing away a portion of the prosthesis along the perforated line of FIG. 14A;

FIG. 15 is a schematic cross-sectional view of another embodiment of a prosthesis disposed over the patient's skull and around the protruding burr hole plug of FIG. 6, the prosthesis including a tapered region extending outwardly from the burr hole plug along the skull;

FIG. 16A is a schematic side view of one embodiment of the prosthesis of FIG. 15;

FIG. 16B is a schematic cross-sectional view of one embodiment of the prosthesis of FIG. 15;

FIG. 16C is a schematic bottom view of one embodiment of the prosthesis of FIG. 15;

FIG. 17A is a schematic first side view of one embodiment of a prosthesis suitable for disposing over an implanted medical device, the prosthesis including an abutting section along the perimeter of the prosthesis body, the abutting section suitable for positioning next to another prosthesis;

FIG. 17B is a schematic bottom view of one embodiment of the prosthesis of FIG. 17A;

FIG. 17C is a schematic second side view of one embodiment of the prosthesis of FIG. 17A rotated 90 degrees from the first side view of FIG. 17A;

FIG. 18A is a schematic top view of two implanted medical devices extending outwardly from a patient's skull and two prostheses disposed over the skull with each prosthesis disposed over a different one of the devices, the prostheses abutting one another along respective abutting sections; and

FIG. 18B is a schematic cross-sectional view of the prostheses of FIG. 18A disposed over the medical devices of FIG. 18A, the prostheses collectively forming a tapered region for smoothing out the protruding implanted medical devices to reduce visibility of bulging caused by the devices.

### DETAILED DESCRIPTION

The present disclosure is directed to the area of implantable medical devices and making and using such devices. The present disclosure is also directed to prostheses for reducing visibility of bulging from implanted medical devices, as well as methods of making and using the prostheses and implantable medical devices.

Suitable implantable electrical stimulation systems include, but are not limited to, a least one lead with one or more electrodes disposed on a distal portion of the lead and one or more terminals disposed on one or more proximal portions of the lead. Leads include, for example, percutaneous leads, paddle leads, cuff leads, or any other arrangement of electrodes on a lead. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,175,710; 8,224,450; 8,271,094; 8,295,944; 8,364,278; 8,391,985; and 8,688,235; and U.S. Patent Applications Publication Nos. 2007/0150036; 2009/0187222; 2009/0276021; 2010/0076535; 2010/0268298; 2011/0005069; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; and 2013/0197602, all of which are incorporated by reference.

Suitable electrical stimulation systems may al so include stimulation lead-less devices, such as implantable microstimulators. Examples of microstimulators are found in, for example, U.S. Pat. Nos. 5,193,539; 5,193,540; 5,324,316; and 8,364,278, all of which are incorporated by reference.

In the discussion below, electrical stimulation systems with percutaneous leads will be exemplified, but it will be understood that the methods and systems described herein are also applicable to other systems, both with and without, leads.

A percutaneous lead for electrical stimulation (for example, deep brain, spinal cord, peripheral nerve, or cardiac-tissue) includes stimulation electrodes that can be ring electrodes, segmented electrodes that extend only partially around the circumference of the lead, or any other type of electrode, or any combination thereof. The segmented electrodes can be provided in sets of electrodes, with each set having electrodes circumferentially distributed about the lead at a particular longitudinal position. A set of segmented electrodes can include any suitable number of electrodes including, for example, two, three, four, or more electrodes. For illustrative purposes, the leads are described herein relative to use for deep brain stimulation, but it will be understood that any of the leads can be used for applications other than deep brain stimulation, including spinal cord stimulation, peripheral nerve stimulation, dorsal root ganglion stimulation, sacral nerve stimulation, or stimulation of other nerves, muscles, and tissues.

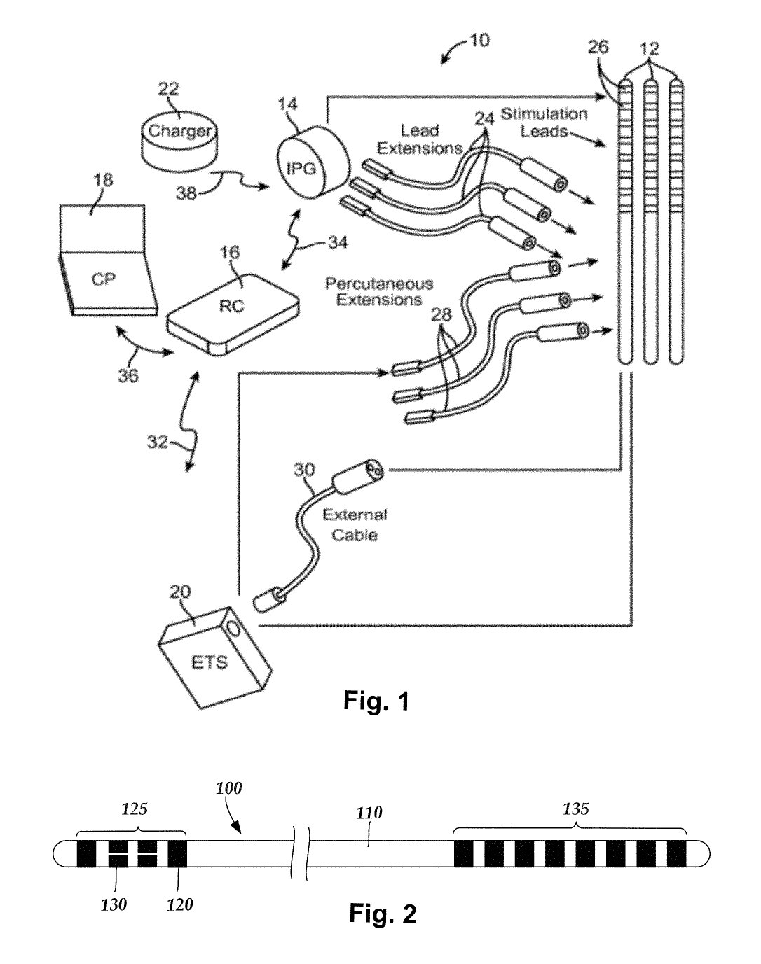

Turning to FIG. 1, one embodiment of an electrical stimulation system 10 includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22.

The IPG 14 is physically connected, optionally, via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The implantable pulse generator can be implanted into a patient's body, for example, below the patient's clavicle area or within the patient's buttocks or abdominal cavity. The implantable pulse generator can have eight stimulation channels which may be independently programmable to control the magnitude of the current stimulus from each channel. In some embodiments, the implantable pulse generator can have more or fewer than eight stimulation channels (e.g., 4-, 6-, 16-, 32-, or more stimulation channels). The implantable pulse generator can have one, two, three, four, or more connector ports, for receiving the terminals of the leads and/or lead extensions.

The ETS 20 may also be physically connected, optionally via the percutaneous lead extensions 28 and external cable 30, to the stimulation leads 12. The ETS 20, which may have similar pulse generation circuitry as the IPG 14, also delivers electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a uni- or bi-directional wireless communications link 32. Once the IPG 14 and neurostimulation leads 12 are implanted, the RC 16 may be used to telemetrically communicate with or control the IPG 14 via a uni- or bi-directional communications link 34. Such communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. Alternately, or additionally, stimulation parameters can be programed via wireless communications (e.g., Bluetooth) between the RC 16 (or external device such as a hand-held electronic device) and the IPG 14.

The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

For purposes of brevity, the details of the RC 16, CP 18, ETS 20, and external charger 22 will not be further described herein. Details of exemplary embodiments of these devices are disclosed in U.S. Pat. No. 6,895,280, which is expressly incorporated herein by reference. Other examples of electrical stimulation systems can be found at U.S. Pat. Nos. 6,181,969; 6,516,227; 6,609,029; 6,609,032; 6,741,892; 7,949,395; 7,244,150; 7,672,734; and 7,761,165; 7,974,706; 8,175,710; 8,224,450; and 8,364,278; and U.S. Patent Application Publication No. 2007/0150036, as well as the other references cited above, all of which are incorporated by reference.

Turning to FIG. 2, one or more leads are configured for coupling with a control module. The term “control module” is used herein to describe a pulse generator (e.g., the IPG 14 or the ETS 20 of FIG. 1). Stimulation signals generated by the control module are emitted by electrodes of the lead(s) to stimulate patient tissue. The electrodes of the lead(s) are electrically coupled to terminals of the lead(s) that, in turn, are electrically coupleable with the control module. In some embodiments, the lead(s) couple(s) directly with the control module. In other embodiments, one or more intermediary devices (e.g., a lead extension, an adaptor, a splitter, or the like) are disposed between the lead(s) and the control module.

Percutaneous leads are described herein for clarity of illustration. It will be understood that paddle leads and cuff leads can be used in lieu of, or in addition to, percutaneous leads. The leads described herein include 8 electrodes (+1 auxiliary electrode in some embodiments). It will be understood that the leads could include any suitable number of electrodes. The leads can include ring electrodes, a distal-tip electrode, and/or one or more segmented electrodes in lieu of, or in addition to one or more ring electrodes. Additionally, the term “elongated member” used herein includes leads (e.g., percutaneous, paddle, cuff, or the like), as well as intermediary devices (e.g., lead extensions, adaptors, splitters, or the like).

FIG. 2 illustrates one embodiment of a lead 100 having a lead body 110 with electrodes 125 disposed at least partially about a circumference of the lead 100 along a distal end portion of the lead and terminals 135 disposed along a proximal end portion of the lead 100. The lead 100 can be implanted near or within the desired portion of the body to be stimulated such as, for example, the brain, spinal cord, or other body organs or tissues. In one example of operation for deep brain stimulation, access to the desired position in the brain can be accomplished by drilling a hole in the patient's skull or cranium with a cranial drill (commonly referred to as a burr), and coagulating and incising the dura mater, or brain covering. The lead 100 can be inserted into the cranium and brain tissue with the assistance of a stylet (not shown). The lead 100 can be guided to the target location within the brain using, for example, a stereotactic frame and a microdrive motor system. In some embodiments, the microdrive motor system can be fully or partially automatic. The microdrive motor system may be configured to perform one or more the following actions (alone or in combination): insert the lead 100, advance the lead 100, retract the lead 100, or rotate the lead 100.

In some embodiments, measurement devices coupled to the muscles or other tissues stimulated by the target neurons, or a unit responsive to the patient or clinician, can be coupled to the implantable pulse generator or microdrive motor system. The measurement device, user, or clinician can indicate a response by the target muscles or other tissues to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

The lead 100 for deep brain stimulation can include stimulation electrodes, recording electrodes, or both. In at least some embodiments, the lead 100 is rotatable so that the stimulation electrodes can be aligned with the target neurons after the neurons have been located using the recording electrodes.

Stimulation electrodes may be disposed on the circumference of the lead 100 to stimulate the target neurons. Stimulation electrodes may be ring-shaped so that current projects from each electrode equally in every direction from the position of the electrode along a length of the lead 100. In the embodiment of FIG. 2, two of the electrodes 125 are ring electrodes 120. Ring electrodes 120 typically do not enable stimulus current to be directed from only a limited angular range around of the lead 100. Segmented electrodes 130, however, can be used to direct stimulus current to a selected angular range around the lead 100. When segmented electrodes 130 are used in conjunction with an implantable pulse generator that delivers constant current stimulus, current steering can be achieved to more precisely deliver the stimulus to a position around an axis of the lead 100 (i.e., radial positioning around the axis of the lead 100). To achieve current steering, segmented electrodes 130 can be utilized in addition to, or as an alternative to, ring electrodes 120.

As described above, the lead 100 includes a lead body 110, terminals 135, and one or more ring electrodes 120 and one or more sets of segmented electrodes 130 (or any other combination of electrodes). The lead body 110 can be formed of a biocompatible, non-conducting material such as, for example, a polymeric material. Suitable polymeric materials include, but are not limited to, silicone, polyurethane, polyurea, polyurethane-urea, polyethylene, or the like. Once implanted in the body, the lead 100 may be in contact with body tissue for extended periods of time. In at least some embodiments, the lead 100 has a cross-sectional diameter of no more than 1.5 mm and may be in the range of 0.5 to 1.5 mm. In at least some embodiments, the lead 100 has a length of at least 10 cm and the length of the lead 100 may be in the range of 10 to 70 cm.

The electrodes 125 can be made using a metal, alloy, conductive oxide, or any other suitable conductive biocompatible material. Examples of suitable materials include, but are not limited to, platinum, platinum iridium alloy, iridium, titanium, tungsten, palladium, palladium rhodium, or the like. Preferably, the electrodes are made of a material that is biocompatible and does not substantially corrode under expected operating conditions in the operating environment for the expected duration of use.

Each of the electrodes can either be used (ON) or unused (OFF). When the electrode is used, the electrode can be used as an anode or cathode and carry anodic or cathodic current. In some instances, an electrode might be an anode for a period of time and a cathode for a period of time.

As described above, deep brain stimulation leads may include one or more sets of segmented electrodes. Segmented electrodes may provide for superior current steering than ring electrodes because target structures in deep brain stimulation are not typically symmetric about the axis of the distal electrode array. Instead, a target may be located on one side of a plane running through the axis of the lead. Through the use of a radially segmented electrode array (“RSEA”), current steering can be performed not only along a length of the lead but also around a circumference of the lead. This provides precise three-dimensional targeting and delivery of the current stimulus to neural target tissue, while potentially avoiding stimulation of other tissue. Examples of leads with segmented electrodes include U.S. Pat. Nos. 8,473,061; 8,571,665; and 8,792,993; U.S. Patent Application Publications Nos. 2010/0268298; 2011/0005069; 2011/0130803; 2011/0130816; 2011/0130817; 2011/0130818; 2011/0078900; 2011/0238129; 2012/0016378; 2012/0046710; 2012/0071949; 2012/0165911; 2012/197375; 2012/0203316; 2012/0203320; 2012/0203321; 2013/0197424; 2013/0197602; 2014/0039587; 2014/0353001; 2014/0358208; 2014/0358209; 2014/0358210; 2015/0045864; 2015/0066120; 2015/0018915; 2015/0051681; U.S. patent application Ser. Nos. 14/557,211 and 14/286,797; and U.S. Provisional Patent Application Ser. No. 62/113,291, all of which are incorporated herein by reference. Segmented electrodes can also be used for other stimulation techniques including, but not limited to, spinal cord stimulation, peripheral nerve stimulation, dorsal root ganglion stimulation, or stimulation of other nerves, muscles, and tissues.

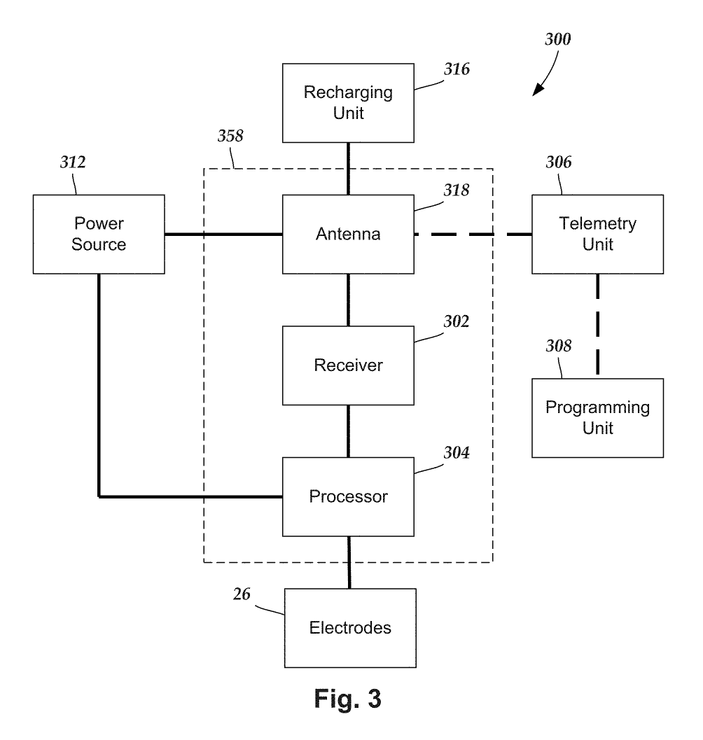

FIG. 3 is a schematic overview of one embodiment of components of an electrical stimulation system 300 including an electronic subassembly 358 disposed within a control module. The electronic subassembly 358 may include one or more components of the IPG. It will be understood that the electrical stimulation system can include more, fewer, or different components and can have a variety of different configurations including those configurations disclosed in the stimulator references cited herein.

Some of the components (for example, a power source 312, one or more antennas 318, a receiver 302, and a processor 304) of the electrical stimulation system can be positioned on one or more circuit boards or similar carriers within a sealed electronics housing of an implantable pulse generator (see e.g., 14 in FIG. 1), if desired. Any power source 312 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 318 or a secondary antenna. In at least some embodiments, the antenna 318 (or the secondary antenna) is implemented using the auxiliary electrically-conductive conductor. The external power source can be in a device that is mounted on the skin of the user or in a unit that is provided near the user on a permanent or periodic basis.

If the power source 312 is a rechargeable battery, the battery may be recharged using the optional antenna 318, if desired. Power can be provided to the battery for recharging by inductively coupling the battery through the antenna to a recharging unit 316 external to the user. Examples of such arrangements can be found in the references identified above. The electronic subassembly 358 and, optionally, the power source 312 can be disposed within a control module (e.g., the IPG 14 or the ETS 20 of FIG. 1).

In one embodiment, electrical stimulation signals are emitted by the electrodes (e.g., electrical array 26 in FIG. 1) to stimulate nerve fibers, muscle fibers, or other body tissues near the electrical stimulation system. The processor 304 is generally included to control the timing and electrical characteristics of the electrical stimulation system. For example, the processor 304 can, if desired, control one or more of the timing, frequency, strength, duration, and waveform of the pulses. In addition, the processor 304 can select which electrodes can be used to provide stimulation, if desired. In some embodiments, the processor 304 selects which electrode(s) are cathodes and which electrode(s) are anodes. In some embodiments, the processor 304 is used to identify which electrodes provide the most useful stimulation of the desired tissue.

Various processors can be used and may be an electronic device that, for example, produces pulses at a regular interval or the processor can be capable of receiving and interpreting instructions from an external programming unit 308 that, for example, allows modification of pulse characteristics. In the illustrated embodiment, the processor 304 is coupled to a receiver 302 which, in turn, is coupled to the optional antenna 318. This allows the processor 304 to receive instructions from an external source to, for example, direct the pulse characteristics and the selection of electrodes, if desired.

In one embodiment, the antenna 318 is capable of receiving signals (e.g., RF signals) from an external telemetry unit 306 which is programmed by the programming unit 308. The programming unit 308 can be external to, or part of, the telemetry unit 306. The telemetry unit 306 can be a device that is worn on the skin of the user or can be carried by the user and can have a form similar to a pager, cellular phone, or remote control, if desired. As another alternative, the telemetry unit 306 may not be worn or carried by the user but may only be available at a home station or at a clinician's office. The programming unit 308 can be any unit that can provide information to the telemetry unit 306 for transmission to the electrical stimulation system 300. The programming unit 308 can be part of the telemetry unit 306 or can provide signals or information to the telemetry unit 306 via a wireless or wired connection. One example of a suitable programming unit 308 is a computer operated by the user or clinician to send signals to the telemetry unit 306.

The signals sent to the processor 304 via the antenna 318 and the receiver 302 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 300 to cease operation, to start operation, to start charging the battery, or to stop charging the battery. In other embodiments, the stimulation system does not include the antenna 318 or receiver 302 and the processor 304 operates as programmed.

Optionally, the electrical stimulation system 300 may include a transmitter (not shown) coupled to the processor 304 and the antenna 318 for transmitting signals back to the telemetry unit 306 or another unit capable of receiving the signals. For example, the electrical stimulation system 300 may transmit signals indicating whether the electrical stimulation system 300 is operating properly or not or indicating when the battery needs to be charged or the level of charge remaining in the battery. The processor 304 may also be capable of transmitting information about the pulse characteristics so that a user or clinician can determine or verify the characteristics.

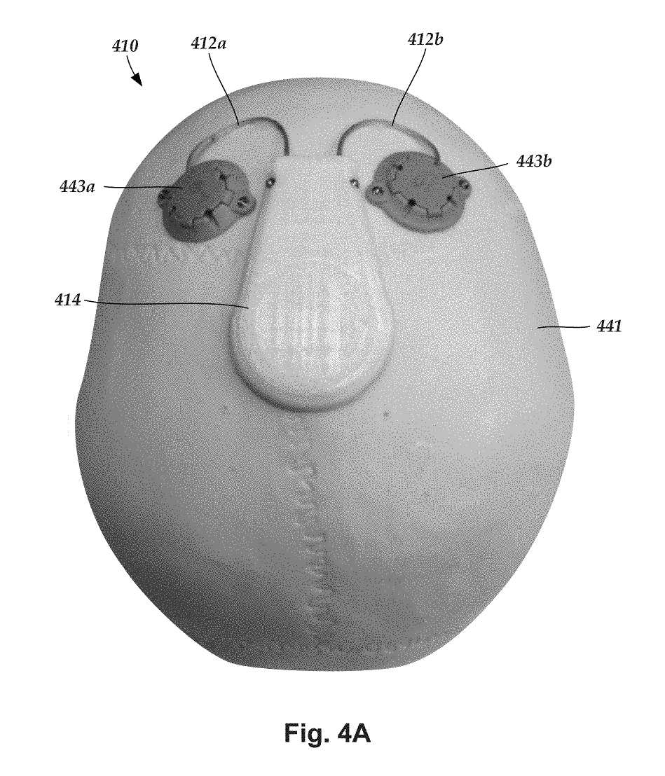

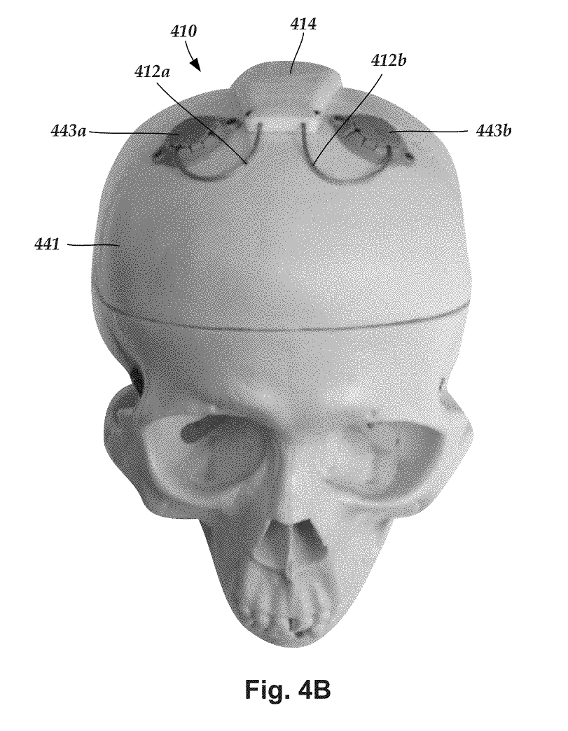

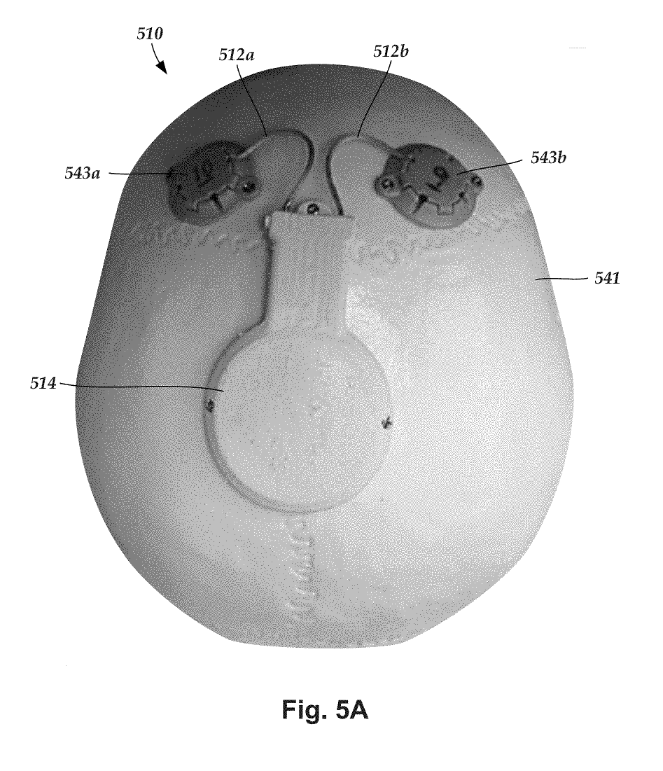

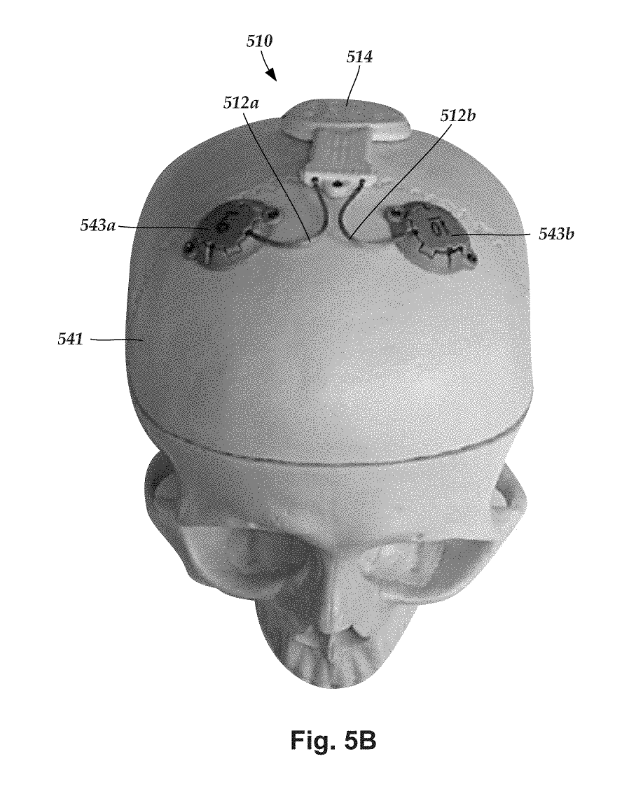

Turning to FIGS. 4A-5B, medical devices implanted into patients may sometimes cause undesired bulging visible along the patient's skin. For example, in the case of deep brain stimulation, one or more leads are typically extended through burr holes drilled into the patient's skull. Burr hole plugs are often disposed in the burr holes to plug the burr holes and retain the lead(s) extending through the burr holes. The burr hole plugs typically extend outwardly from an outer surface of the patient's skull.

When the patient's scalp is repositioned over the skull following a lead implantation procedure, portions of the burr hole plugs extending outwardly from the patient's skull may form bulges along the patient's head that are visible through the skin. Many patients consider the bulging to be unsightly. Moreover, the bulging can contribute to undesirable irritation, and even erosion, of patient tissue.

In some instances, the control module (e.g., IPG 14) coupled to the one or more leads is implanted at a location remote from the patient's head, such as the patient's clavicle area. Alternately, the control module may be mounted along an outer surface of the patient's skull. When the control module is skull-mounted, the control module typically extends outwardly from the patient's skull by an amount sufficient to form a bulge in the patient's head that is visible through the patient's scalp following the lead implantation procedure. In some instances, a medical practitioner may carve out a section of skull large enough to position the control module partially within the carved-out region to reduce the distance that the control module extends outwardly from the patient's skull. However, such a technique may be time-consuming and tedious for the medical practitioner, and invasive for the patient. Additionally, even when a skull-mounted control module is partially inset into the patient's skull, the control module may still extend outwardly from the skull by an amount sufficient to form a visible bulge.

FIGS. 4A-5B show several embodiments of an implantable electrical stimulation system with leads extending through burr holes formed in a skull. Burr hole plugs are disposed in the burr holes. A skull-mounted control module (IPG) is shown coupled to the leads. One example of an implantable electrical stimulation system with leads extending through burr holes formed in a skull and a skull-mounted IPG is found in U.S. Patent Application No. 62/585,405, which is incorporated by reference. As mentioned above, the IPG can, optionally, be implanted in other bodily locations instead of the skull including, for example, the clavicle.

FIG. 4A shows, in top view, one embodiment of an electrical stimulation system 410 that includes a control module 414 disposed along an outer surface of a skull 441. FIG. 4B shows the electrical stimulation system 410 and skull 441 in front view. Two leads 412a, 412b extend from the control module 414 and into the skull 441 via burr holes, over which burr hole plugs 443a, 443b, respectively, are disposed.

FIG. 5A shows, in top view, another embodiment of an electrical stimulation system 510 that includes a control module 514 disposed along an outer surface of a skull 541. FIG. 5B shows the electrical stimulation system 510 and skull 541 in front view. Two leads 512a, 512b extend from the control module 514 and into the skull 541 via burr holes, over which burr hole plugs 543a, 543b, respectively, are disposed.

As shown in FIGS. 4A-5B, the burr hole plugs and the control module each extend radially outward from the skull. Accordingly, when the patient's scalp is subsequently repositioned over the skull, the portions of the burr hole plugs and control module extending outwardly from the skull may form undesirable bulges visible through the patient's skin. In addition to being unsightly, the bulging may also cause the patient to experience skin erosion and/or irritation along and around the bulges.

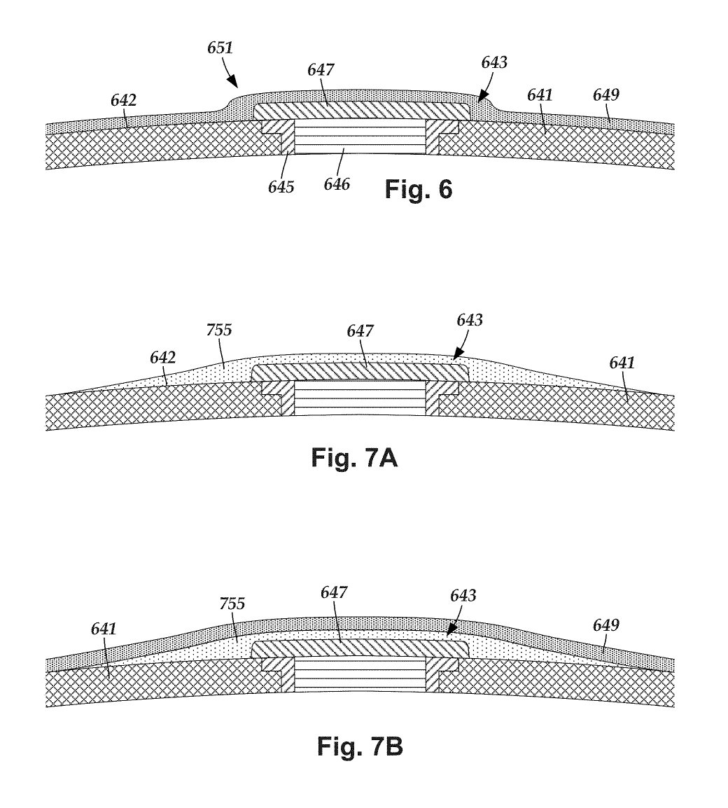

FIG. 6 shows, in schematic cross-section view, one embodiment of a burr hole plug 643 disposed in a burr hole formed in a patient's skull 641. The burr hole plug 643 includes a housing 645 forming a bore 646 extending through the skull, and a cover assembly 647 disposed over the housing and bore. The housing 645 may, optionally, include a retainer (not shown in FIG. 6) for receiving and retaining a lead (not shown in FIG. 6) extending through the skull via the burr hole. The cover assembly 647 is disposed over the housing 645 and may define an optional notch or aperture (not shown in FIG. 6) configured to enable a lead to extend through the cover assembly. Note that in FIG. 6, and in other figures, inset regions of cross-sectional views (such as bore 646) include horizontal lines, for clarity of illustration.

In some embodiments, the cover assembly 647 includes a cap. In some embodiments, the cover assembly 647 includes a flap. In some embodiments, the cover assembly 647 includes one or more fasteners for fastening the cover assembly to the skull. The cover assembly 647 may, optionally, include one or more coatings, an overmold, or both. The overmold may be disposed over the cap, flap, coating, or combination thereof. In some embodiments, one or more coatings are disposed over the overmold.

At least a portion of the cover assembly 647 typically extends outwardly from an outer surface 642 of the skull 641. As shown in FIG. 6, when the patient's scalp 649 is repositioned over the patient's scalp following an implantation procedure, the portion of the burr hole plug (e.g., the cover assembly 647) extending outwardly (i.e., protruding) from the patient's skull can alter the natural contouring of the patient's skin extending over the skull and form a bulge 651 visible through the patient's scalp. In some embodiments, the entire cover assembly extends outwardly from the patient's skull. In other embodiments, only a portion of the cover assembly extends outwardly from the patient's skull. In some embodiments, one or more other portions of the burr hole plug, such as the housing, also at least partially extend outwardly from the patient's skull.

As described herein, a prosthesis is used to improve one or more of patient cosmetic outcome, skin erosion, or skin irritability following implantation of a medical device into a patient by tapering bulging caused by the implanted medical device. The prosthesis at least partially covers (e.g., at least partially surrounds, at least partially extends over top of, or the like) one or more portions of the implanted medical device (or one or more protruding portions thereof) and functions to alter the contouring of the overlying skin caused, at least in part, by at least a portion of the implanted medical device, thereby forming a smoother transition between the portions of the patient's skin disposed over the implanted medical device and one or more surrounding portions of the patient's skin.

In other words, the prosthesis tapers a bulge caused by a protruding implanted medical device to facilitate blending in of the medical device with its surrounding environment. By blending the implanted medical device in with its surroundings, the patient may experience less visible, or noticeable, bulging. Alternately, or additionally, by blending the implanted medical device in with its surroundings the patient may experience less skin erosion and irritation than the patient would otherwise experience by overlying skin rubbing along hot spots created along portions of the skin by the protruding implanted device.

In some embodiments, the prosthesis is configured to at least partially cover a portion of an implanted medical device extending radially outward from the patient's skull including, for example, a burr hole plug, a lead, a control module (IPG) of an electrical stimulation system, or the like or combinations thereof. In the case of a control module, smoothing out the bulge may obviate the need to create a depression in the patient's skull to partially inset the control module. Obviating the need to create the depression may save reduce the cost, complexity, invasiveness, and duration of an implantation procedure without unduly sacrificing the cosmetic outcome for the patient.

For illustrative purposes, the prosthesis will be described as being used in conjunction with burr hole plugs and IPGs (see e.g., FIGS. 4A-5B) of an electrical stimulation system. It will be understood, however, that the prosthesis can be used in conjunction with other implanted medical devices that may form bulges along patient skin. The implanted medical devices can be stimulating or non-stimulating and can be implanted along any suitable portion of the body. For example, in some embodiments the prosthesis is used in conjunction with one or more peripheral stimulation systems, such as a microstimulator, disposed along one or more other portions of a patient's body including, for example, an arm, leg, neck, torso, or abdomen of the patient. It will also be understood that the location of implantation and the size and shape of the implanted medical device can influence one or more features of the prosthesis, such as the size or shape of the prosthesis along at least one axis.

Turning to FIGS. 7A-14B, in some embodiments the prosthesis is configured for disposing over and around a medical device implanted beneath a patient's scalp and at least partially protruding from the patient's skull. The prosthesis, in some embodiments, defines a cavity configured to receive one or more protruding portions the implanted medical device, or even the entire implanted medical device.

FIG. 7A shows, in schematic cross-sectional view, one embodiment of a prosthesis 755 disposed over top of the burr hole plug 643 and along a portion of the outer surface 642 of the patient's skull 641 surrounding the burr hole plug 643. FIG. 7B shows, in schematic cross-sectional view, one embodiment of the patient's scalp 649 disposed over the prosthesis 755 and surrounding outer surface 642 of the patient's skull 641.

As shown in FIGS. 7A-7B, the prosthesis 755 can be configured for implanting over the patient's skull and beneath the patient's scalp. The prosthesis is shown positioned over, and covering, the portion of the implanted medical device extending outwardly from the skull by an amount sufficient to cause bulging along the scalp when the scalp is laid over top of the skull after implantation. The prosthesis functions to taper the contouring of the protrusion caused by the implanted medical device, thereby reducing visibility of bulging caused by the implanted device.

In some embodiments, the prosthesis 755 effectively replaces the contour of the portion of the implanted medical device extending outwardly from the skull (see e.g., FIG. 6) with a more tapered contour (see e.g., FIG. 7B) that more closely blends in with the patient's natural external contouring over the region of implantation, thereby reducing visibility of the implanted medical device, as well as reducing skin erosion and irritability caused by the protruding device, after implantation.

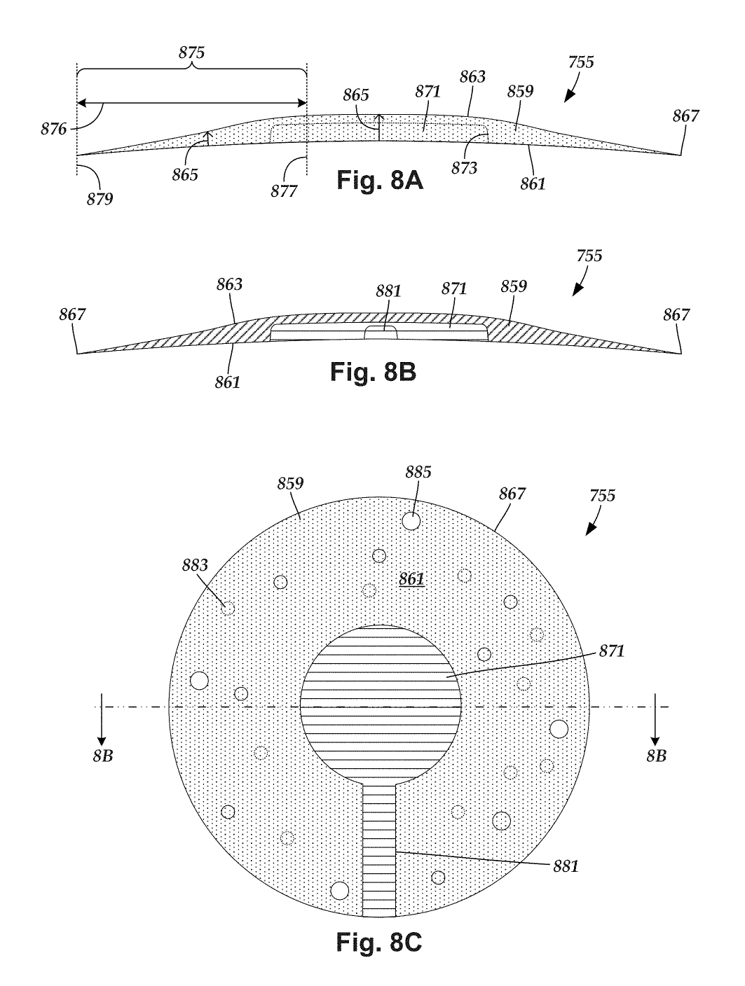

FIG. 8A shows, in schematic side view, one embodiment of the prosthesis 755. FIG. 8B shows one embodiment of the prosthesis 755 in schematic cross-sectional view. FIG. 8C shows one embodiment of the prosthesis 755 in schematic bottom view. The prosthesis 755 includes a body 859 with a first major surface 861, an opposing second major surface 863, a height 865, and an outer perimeter 867.

The prosthesis shown in FIGS. 8A-8C is configured for implanting in a patient with the first major surface 861 positioned against the patient's skull (either directly over the patient's skull or over one or more layers of tissue) and the second major surface 863 positioned against the patient's scalp (either directly beneath the patient's scalp or under one or more layers of materials disposed between the prosthesis and the patient's scalp).

In at least some embodiments, the body is substantially flat. In some embodiments, the first major surface has a concave shape along at least two different non-parallel dimensions. In some embodiments, the concave shape of the first major surface is configured to lay flat against (e.g., conform to) a convex outer portion of the patient's skull.

In some embodiments, the second major surface has a convex shape along at least two different non-parallel dimensions. It may be advantageous for the second major surface to be convex along at least two different non-parallel dimensions to provide a desired contour upon which the scalp is laid against, such as a contour that more closely matches the natural contouring of the patient's head along the implantation region prior to implantation of the medical device than were the scalp to be laid directly against the protruding implanted medical device. In other words, it may be advantageous for the second major surface to be convex along at least two different non-parallel dimensions to provide a contour upon which the scalp is laid against that blends in with the shape of the patient's head at and around the region of implantation.

The prosthesis 755 includes a cavity 871 defined along the first major surface 861. The cavity 871 is defined by an inner wall 873 extending into the body 859 from the first major surface 861. The cavity 871 is configured to receive and cover the protruding portion of an implanted medical device, such as the protruding portion of the burr hole plug (643 in FIGS. 6-7B), when the prosthesis is disposed against the patient's skull.

The height (i.e., thickness), is the shortest distance to the second major surface from a given location along the first major surface. When the prosthetic is disposed along a skull, the height is perpendicular to the outer surface of the skull. In some embodiments, the height of the body is largest at the center of the body. In other embodiments, the height of the body is largest along a portion of the body that is peripheral to the center of the body. In the embodiment shown in FIGS. 8A-8C, the height 865 is greater than the distance that the implanted medical device extends outwardly from the patient's skull so that the first major surface of the prosthesis lies flat against the patient's skull while the protruding portion of the implanted medical device is disposed entirely in the cavity of the prosthesis.

The body 859 can be formed from any biocompatible material suitable for implantation, including metals, alloys, polymers, plastics, resins, or the like. In some embodiments, the body includes one or more of silicone, polyether ether ketone, or Titanium. In some embodiments, the body consists of one or more of silicone, polyether ether ketone, or Titanium. In some embodiments, the body consists essentially of one or more of silicone, polyether ether ketone, or Titanium. The prosthesis can be formed from any suitable technique including, for example, injection molding, 3D printing, or the like.

The body 859 further includes at least one tapered region 875 that tapers the height of the body as the body extends peripherally towards the outer perimeter to facilitate blending in of the implanted medical device with a desired contour (e.g., natural, pre-implantation contour; or new contour) over the region of implantation. The tapered region has a length 876 defined by the shortest distance extending radially from an inner boundary 877 to an outer boundary 879, where the inner boundary is medial to the outer boundary along the body. In some embodiments, the outer boundary 879 is the outer perimeter 867 of the body.

The length of the tapered region may be based on any number of different factors, such as varying curvatures in different directions at the region of implantation, proximity to one or more features (e.g., other implanted medical devices). In some embodiments, the tapered region has a constant length. In other embodiments, the tapered region has a variable length. In some embodiments, the length of the tapered region is no less than 1 cm, 2 cm, 3 cm, 4 cm, 5 cm, 6 cm or more.

In some embodiments, the inner boundary 877 of the tapered region is medial to the inner wall 873 of the cavity. In some embodiments, the inner boundary 877 is lateral to the inner wall 873 of the cavity. In some embodiments, the inner boundary 877 is even with the inner wall 873 of the cavity. In some embodiments, a first portion of the inner boundary 877 is medial to the inner wall 873 and a second portion of the inner boundary 877 is lateral to the inner wall 873. In some embodiments, the entire body tapers. In some embodiments, the second major surface is dome-shaped.

In some embodiments, the body includes a single tapered region. In some embodiments, the body includes a single tapered region that extends along the entire perimeter. In some embodiments, the body includes multiple tapered regions, with equal or unequal lengths to one another. The tapered region(s) can collectively extend along all, or only a portion, of the perimeter of the body. In some embodiments, the tapered region(s) extend(s) along at least 50%, 60%, 70%, 80%, 90% of the outer perimeter.

As shown in FIG. 8C, in at least some embodiments the body defines one or more channels 881 inset to, and open along, the first major surface 861. The channels, in some embodiments, extend from the cavity 871 to the outer perimeter 867 of the body. It may be advantageous to define one or more channels to accommodate one or more additional medical devices. For example, when the cavity 871 is configured to receive a portion of a burr hole plug or a skull-mounted IPG, the one or more channels may be used to accommodate one or more leads extending to/from the burr hole plug or skull-mounted IPG. In some instances, the one or more channels are configured to receive the one or more leads while enabling the first major surface to remain flush against the patient's skull.

In some embodiments, the body defines one or more surface features 883 for promoting tissue ingrowth. The surface feature(s) can be disposed along the first major surface 861, the second major surface 863, or both. In FIG. 8C, surface features are shown disposed along both the first major surface and the second major surface. In at least some embodiments, the surface features are formed as apertures extending into the body into which patient tissue can grow into after implantation. The surface features can be formed in any shape or size suitable for promoting tissue ingrowth. In FIG. 8C, the surface features are shown as being round, for clarity of illustration.

In some embodiments, the body is affixable to the skull, the scalp, the protruding portion of the implanted medical device, or some combination thereof, to facilitate prevention of movement of the prosthesis relative to the patient or the implanted medical device. In some embodiments, the body includes one or more retention features, such as one or more fastener apertures 885, configured for receiving a fastener, such as a screw, pin, or the like. In at least some embodiments, an adhesive is used in lieu of, or in addition to, one or more fasteners. The adhesive can be applied to the first major surface, the second major surface, the cavity, or any combination thereof to facilitate affixing of the prosthesis to the skull, the scalp, the medical device, or any combination thereof.

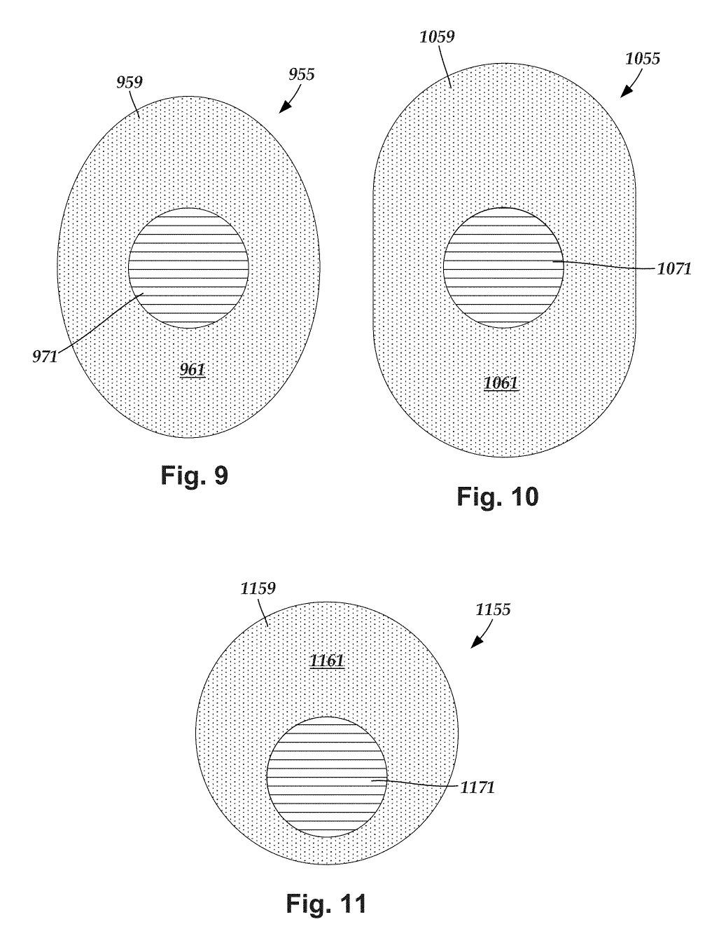

Turning to FIGS. 9-10, the prosthesis can have any suitable shape for facilitating blending in the implanted device with the patient's external bodily contours over the implantation region. In FIGS. 8A-8C, the first major surface is shown as being round. Other shapes are contemplated including, for example, oval-shaped, capsule-shaped, as well as other geometric and non-geometric shapes. The outer perimeter can include one or more straight portions, one or more curved portions, or both.

FIG. 9 shows, in schematic bottom view, one embodiment of a prosthesis 955 with an oval-shaped body 959 and a cavity 971 defined along a first major surface 961 of the body 959. FIG. 10 shows, in schematic bottom view, one embodiment of a prosthesis 1055 with a capsule-shaped body 1059 and a cavity 1071 defined along a first major surface 1061 of the body 1059.

Turning to FIG. 11, in at least some embodiments the prosthesis defines a cavity that is not positioned at the center of the prosthesis body when viewed in bottom view. FIG. 11 shows, in schematic bottom view, one embodiment of a prosthesis 1155 with a cavity 1071 defined along a first major surface 1161 of a body 1159, where the cavity 1071 is positioned off-center of the first major surface 1161.

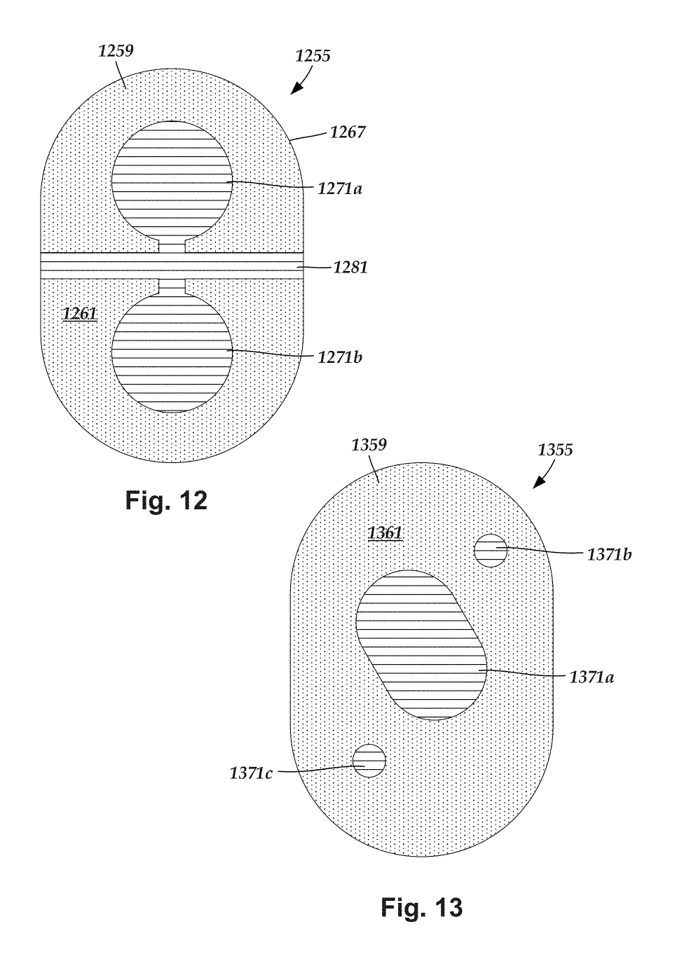

Turning to FIGS. 12-13, in at least some embodiments the prosthesis defines multiple cavities configured to receive multiple protrusions of one or more implanted medical devices. The multiple cavities can all be defined along the first major surface, or one or more of the multiple cavities can be disposed along the second major surface. It may be an advantage to include multiple cavities for receiving medical devices when, for example, multiple medical devices (or multiple portions of the same medical device) are implanted too close together to enable a different prosthesis to be positioned over each of the medical devices (portions of the medical device) without overlapping the tapered regions of the other prostheses.

FIG. 12 shows, in schematic bottom view, one embodiment of a prosthesis 1255 with a body 1259 that defines two cavities 1271a, 1271b along the first major surface 1261. In FIG. 12, the cavities are each shown having the same shape and size. In some embodiments, one or more channels, such as channel 1281, are defined along the first major surface 1261 of the body to accommodate a medical device. In some embodiments, one or more of the channels extend between one or more of the cavities and an outer perimeter 1267 of the body 1259. In some embodiments, one or more of the channels extend between the cavities. In some embodiments, one or more channels extend between the cavities and the outer perimeter. In some embodiments, multiple channels are configured to intersect with one another to provide different routing options (e.g., for one or more leads).

In at least some embodiments, it may be advantageous to define one or more cavities with a non-round transverse shape to accommodate a medical device (or portion thereof) that has a non-round transverse shape. FIG. 13 shows, in schematic bottom view, one embodiment of a prosthesis 1255 with a body 1359 defining three cavities 1371a-c defined along a first major surface 1361 of the body 1359, where one of the cavities 1371a has a non-round transverse shape. It will be understood that the prosthesis may define any suitable number of cavities for receiving any suitable number of medical devices (or portions thereof) including, for example, one, two, three, four, five, six, seven, eight, nine, ten, fifteen, twenty, or more cavities.

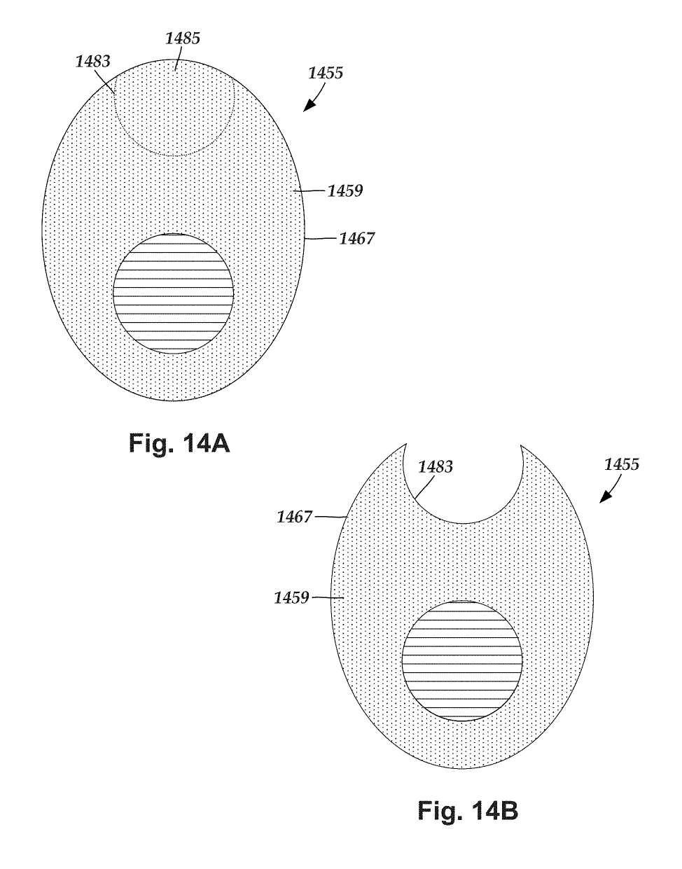

Turning to FIGS. 14A-14B, in some embodiments the outer perimeter of the prosthesis can be altered or customized, as needed. In some instances, it may be desirable to remove a portion of the body to improve the fit of the prosthesis at the region of implantation (e.g., reduce or prevent bunching or folding or undesirably overlapping with patient tissue or an implanted device). In some embodiments, the body is formed, at least in part, from a material that can be cut or torn, as needed. In some embodiments, the body includes one or more perforated lines to facilitate cutting or tearing of the body.

FIG. 14A shows, in schematic bottom view, one embodiment of a prosthesis 1455 having a body 1459 with a perimeter 1467. A perforated line 1483 is formed along the body 1459. The perforated line can be formed along any suitable portion of the body. In FIG. 14A, the perforated line 1483 is shown extending from a first location along the perimeter to a second location along the perimeter. Consequently, when the body is cut or torn along the entire perforated line, a section 1485 is removed from the remaining portions of the body.

FIG. 14B shows, in schematic bottom view, one embodiment of the body 1459 of the prosthesis 1455 cut or torn along the perforated line 1483. In FIG. 14B, section 1485 is removed from the body. In some embodiments, tearing (or ripping, cutting, or the like) the body does not result in removal of a section of the body.

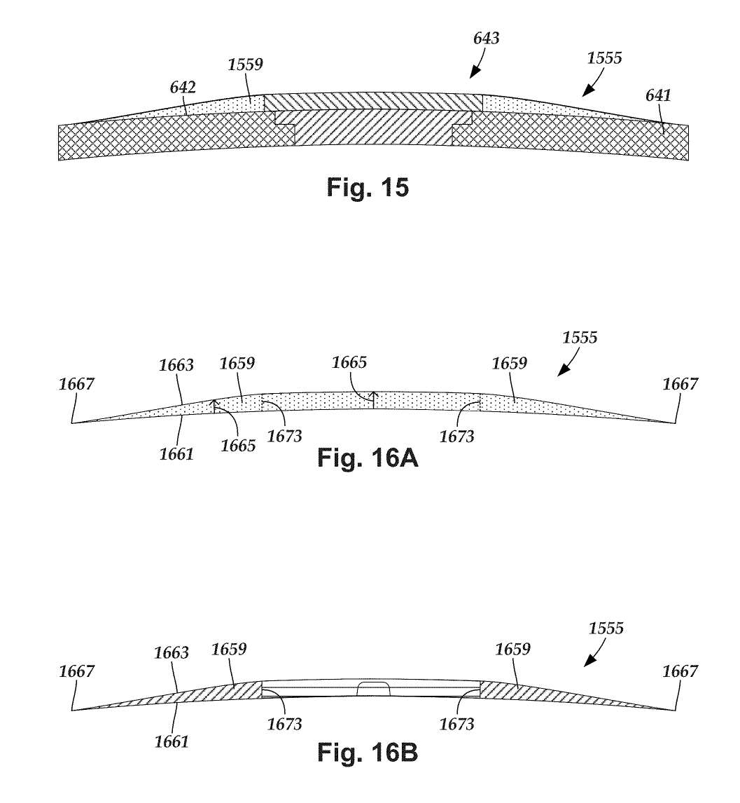

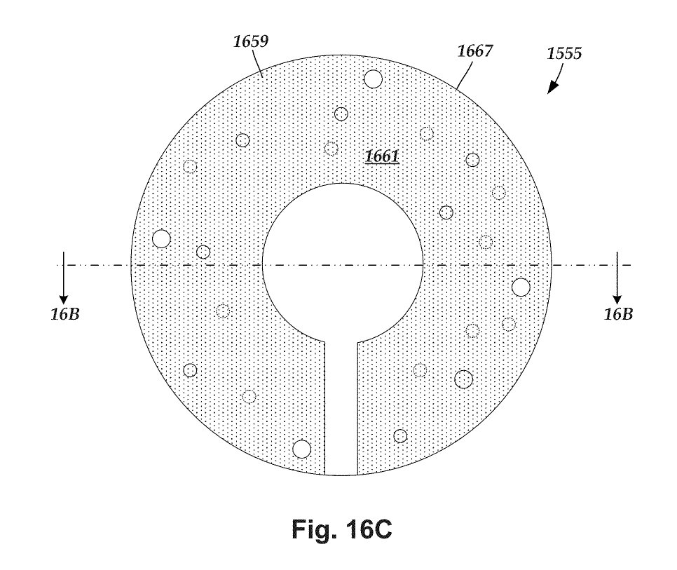

Turning to FIGS. 15-16C, in some embodiments the inner walls of the cavity extend entirely between the first to second major surfaces. When the inner walls of the cavity extend entirely between the first and second major surfaces, the prosthesis can be disposed over an implanted medical device with a portion of the implanted medical device extending outwardly from the patient's skull encircled by the inner walls so that the prosthesis surrounds, but does not completely extend over top of, the portion of the implanted medical device extending outwardly from the patient's skull. In some embodiments, the inner walls of the prosthesis surround, and do not extend over top of, the portion of the implanted medical device extending outwardly from the patient's skull.

FIG. 15 shows, in schematic cross-sectional view, one embodiment of a prosthesis 1555 disposed over the burr hole plug 643, as well as a portion of the outer surface 642 of the patient's skull 641 surrounding the burr hole plug 643. FIG. 16A shows, in schematic side view, one embodiment of the prosthesis 1555. FIG. 16B shows one embodiment of the prosthesis 1555 in schematic cross-sectional view. FIG. 16C shows one embodiment of the prosthesis 1555 in schematic bottom view. The prosthesis 1555 includes a body 1659 with a first major surface 1661, an opposing second major surface 1663, a height 1665, and an outer perimeter 1667.

The prosthesis shown in FIGS. 15-16C is similar to the prostheses of the embodiments shown FIGS. 7A-14B (e.g., with one or more optional tapered regions, channels, surface features, fastener apertures, tearaway portions, and the like), but instead of defining a cavity that completely covers the protruding portion of the device, the prosthesis 1555 includes a central opening 1687 that is defined by an inner wall 1673 and that extends completely through the body 1659 from the first major surface 1661 to the second major surface 1663. The central opening 1687 receives and surrounds the protruding portion of the device but does not completely cover the device. Note that the central opening 1687 need not be defined along the center of the body. The central opening 1687 can be defined along any portion of the body within the outer perimeter 1667.

The height (i.e., thickness) can be any suitable length relative to a distance that the implanted medical device extends outwardly from the skull. In some embodiments, the height is smaller than a distance that the implanted medical device extends outwardly from the skull. In some embodiments, the height is larger than a distance that the implanted medical device extends outwardly from the skull. In some embodiments, the height is equal to a distance that the implanted medical device extends outwardly from the skull.

The prosthesis 1555 can have any suitable shape for facilitating blending in the implanted device with the patient's external bodily contours over the implantation region. The central opening can be positioned at the center of the prosthesis body when viewed in bottom view, or along any other suitable portion of the body. In at least some embodiments the prosthesis 1555 defines multiple central openings configured to receive multiple protrusions of one or more implanted medical devices. The multiple cavities can all be defined along the first major surface, or one or more of the multiple cavities can be disposed along the second major surface. The central opening can have any shape suitable for surrounding a protruding portion of an implanted medical device.

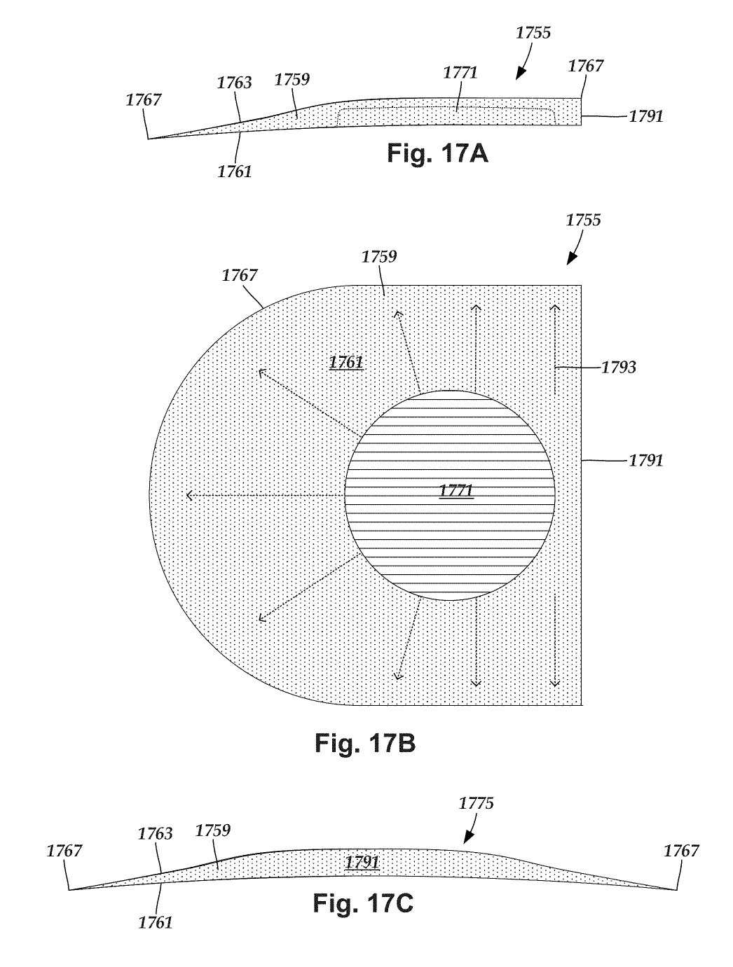

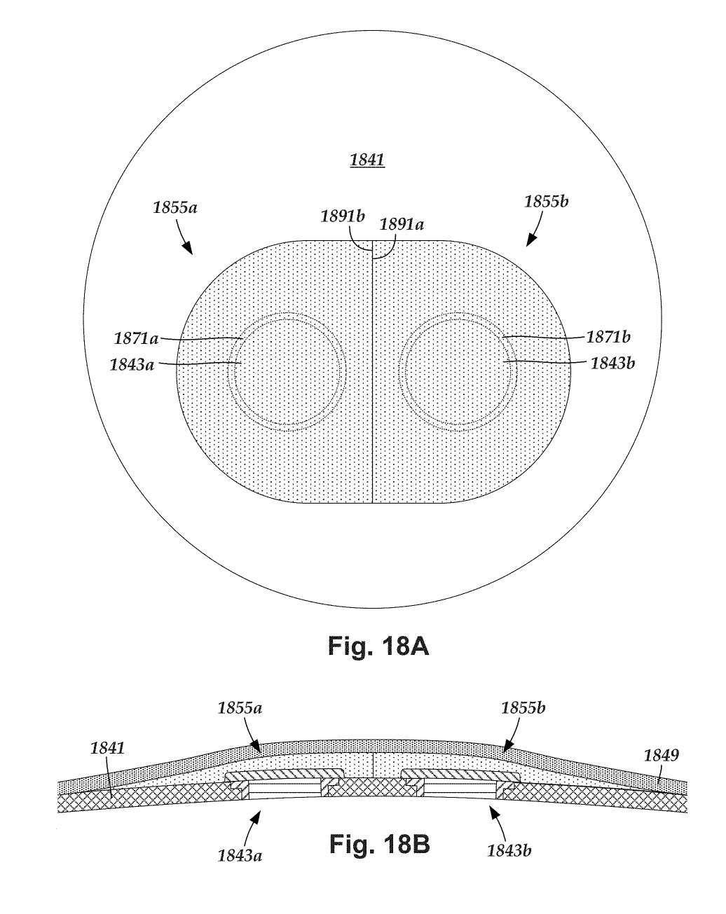

Turning to FIGS. 17A-18B, in some embodiments is may be desirable to use multiple prostheses together to collectively smooth out multiple protruding medical devices. For example, FIGS. 4A-5B show two burr hole plugs which may, in some instances, be positioned close enough together that either a single prosthesis with multiple cavities (or central openings) are utilized (see e.g., FIG. 12), or multiple prostheses are used to collectively smooth out a region of implantation with multiple protruding devices, or portions of devices.

FIG. 17A shows, in a first schematic side view, one embodiment of a prosthesis 1755. FIG. 17B shows one embodiment of the prosthesis 1755 in schematic bottom view. FIG. 17C shows one embodiment of the prosthesis 1755 in a second schematic side view rotated 90° from the first side view of FIG. 17A. The prosthesis 1755 includes a body 1759 with a first major surface 1761, an opposing second major surface 1763, a cavity 1771, and an outer perimeter 1767.

The outer perimeter 1767 includes an abutting section 1791 configured to align and abut with a corresponding abutting section of another prosthesis. In FIGS. 17A-17B, the abutting section is shown as being a straight edge. Other abut-able, mate-able, or interlock-able abutting sections can be implemented including, for example, a saw-tooth edge, sine-wave edge, square-wave edge, or the like.

When multiple prostheses are used to collectively smooth out a region of implantation, it may be advantageous for the directions of tapering of the multiple prostheses to coordinate with one another to collectively create a desired contour and prevent undesired dips, depressions, or the like, caused by abutting the tapered edges of multiple prostheses with one another. As an example, FIG. 17B includes arrows, such as arrow 1793, showing the direction of tapering. In the embodiment shown in FIGS. 17A-17C, the body does not taper towards the abutting section 1791.

FIG. 18A shows two prostheses 1855a and 1855b arranged side-by-side with one another along a skull 1841 with respective abutting sections 1891a, 1891b of the prostheses abutting one another. The prosthesis 1855a defines a cavity 1871a disposed over a burr hole plug 1843a, and the prosthesis 1855b defines a cavity 1871b disposed over a burr hole plug 1843b. Collectively, the prostheses 1855a, 1855b form a tapered contour that blends in with the patient's natural external contouring over the regions of implantation, thereby reducing visibility of the implanted medical devices, as well as reducing skin erosion and irritability caused by the protruding devices, after implantation.

FIG. 18B shows, in schematic cross-sectional view, one embodiment of the patient's scalp 1849 disposed over the prostheses 1855a, 1855b and the patient's skull 1841. As shown in FIG. 18B, the prostheses 1855a, 1855b collectively form a tapered contour that blends in with the patient's natural external contouring over the regions of implantation.

The above specification and examples provide a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. A cranial prosthesis for implanting over a portion of a patient's skull, the cranial prosthesis comprising:
a body comprising
a first major surface configured for positioning against the patient's skull upon implantation of the cranial prosthesis,
a second major surface opposite to the first major surface and forming a contour along which the patient's scalp is disposed against upon implantation of the cranial prosthesis,
an outer perimeter defining a boundary between the first and second major surfaces,
a cavity defined along a portion of the first major surface, the cavity configured for receiving and covering a portion of a medical device extending outwardly from the patient's skull, and
a tapered region extending radially outward along the body toward the outer perimeter, the tapered region tapering the contour of the second major surface to reduce visibility of bulging along the patient's scalp caused by the portion of a medical device extending outwardly from the patient's skull when the prosthesis is disposed over the implanted medical device.

2. The cranial prosthesis of claim 1, wherein the tapered region extends along no less than 50% of the outer perimeter of the body.

3. The cranial prosthesis of claim 1, wherein the tapered region extends radially outward along the body from an inner boundary toward the outer perimeter, the inner boundary positioned radially inward from the outer perimeter by a distance of no less than 3 cm.

4. The cranial prosthesis of claim 1, wherein the body defines at least one attachment aperture configured for receiving a fastener for fastening the cranial prosthesis to the skull.

5. The cranial prosthesis of claim 1, wherein the first major surface defines a channel extending between the cavity and the outer perimeter, the channel configured to receive a portion of an electrical stimulation lead extending along the skull.

6. The cranial prosthesis of claim 1, wherein the body defines one or more surface features configured for facilitating tissue ingrowth.

7. The cranial prosthesis of claim 1, wherein the tapered region extends to the outer perimeter of the body.

8. The cranial prosthesis of claim 1, wherein the second major surface is convex along at least two nonparallel axes.

9. The cranial prosthesis of claim 1, wherein the first major surface is concave along at least two nonparallel axes.

10. The cranial prosthetic of claim 1, wherein the body comprises a height defining a smallest distance to the second major surface from a given location along the first major surface, and wherein the height of the body gets smaller along the tapered region moving towards the outer perimeter.

11. A method of implanting a medical device within a patient, the method comprising:
positioning the medical device on or in the patient's skull with a portion of the medical device extending outwardly from the skull;
disposing the cranial prosthesis of claim 1 over the skull with the portion of the medical device extending outwardly from the skull disposed in the cavity of the cranial prosthesis; and
covering the skull and cranial prosthesis with the patient's scalp;
wherein the contour of the second major surface along the tapered region of the cranial prosthesis reduces visibility through the scalp of bulging caused by the portion of the medical device extending outwardly from the skull.

12. The method of claim 11, wherein positioning the medical device on or in the patient's skull with a portion of the medical device extending outwardly from the skull comprises
forming a burr hole in the patient's skull; and
disposing a burr hole plug in the burr hole, the burr hole plug at least partially extending outwardly from the skull.

13. The method of claim 11, wherein positioning the medical device on or in the patient's skull with a portion of the medical device extending outwardly from the skull comprises mounting a pulse generator to the patient's skull with at least a portion of the pulse generator extending outwardly from the skull.

14. An implantable medical device kit comprising:
an electrical stimulation system comprising
a burr hole plug configured to dispose in a burr hole formed in a skull of a patient, the burr hole plug comprising a cover assembly disposed over the burr hole and at least partially extending outwardly from an outer surface of the patient,
an electrical stimulation lead configured to extend through the burr hole cover and into the burr hole from a location at least partially external to the patient's skull, and
a pulse generator configured to generate stimulation energy and coupleable with the electrical stimulation lead, the pulse generator configured for implanting into the patient at a location at least partially external to the patient's skull; and
the cranial prosthesis of claim 1, the cranial prosthesis configured to reduce visibility of bulging along the scalp caused by the portion of the electrical stimulation system extending outwardly from the patient's skull when the cranial prosthesis is disposed over a portion of the electrical stimulation system.

15. The medical device kit of claim 14, wherein the cranial prosthesis is to reduce visibility of bulging along the scalp caused by the burr hole plug when the cranial prosthesis is disposed over the burr hole plug.

16. The medical device kit of claim 14, wherein the pulse generator is mounted on the patient's skull and extends outwardly from the outer surface of the patient skull, and wherein the cranial prosthesis is configured to reduce visibility of bulging along the scalp caused by the pulse generator when the cranial prosthesis is disposed over the pulse generator.

17. A cranial prosthesis for implanting over a portion of a patient's skull, the cranial prosthesis comprising:
a body comprising
a first major surface configured for positioning against the patient's skull upon implantation of the cranial prosthesis,
a second major surface opposite to the first major surface and forming a contour along which the patient's scalp is disposed against upon implantation of the cranial prosthesis,
an outer perimeter defining a boundary between the first and second major surfaces,
an inner wall forming a central opening extending between the first and second surfaces, the inner walls configured for receiving a portion of an implanted medical device extending outwardly from the patient's skull, and
a tapered region extending radially outward along the body toward the outer perimeter, the tapered region tapering the contour of the second major surface to reduce visibility of bulging along the patient's scalp caused by the portion of a medical device extending outwardly from the patient's skull when the prosthesis is disposed over the implanted medical device.

18. The cranial prosthesis of claim 17, wherein the body comprises a height defining a smallest distance to the second major surface from a given location along the first major surface, and wherein the height of the body along the inner wall is no greater than a distance that the portion of the implanted medical device extending outwardly from the patient's skull extends outwardly from the patient's skull.

19. A method of implanting a medical device within a patient, the method comprising:
positioning the medical device on or in the patient's skull with a portion of the medical device extending outwardly from the skull;
disposing the cranial prosthesis of claim 17 over the skull with the portion of the medical device extending outwardly from the skull disposed in the central opening of the cranial prosthesis; and
covering the skull and cranial prosthesis with the patient's scalp;
wherein the contour of the second major surface along the tapered region of the cranial prosthesis reduces visibility through the scalp of bulging caused by the portion of the medical device extending outwardly from the skull.

20. An implantable medical device kit comprising:
an electrical stimulation system comprising
a burr hole plug configured to dispose in a burr hole formed in a skull of a patient, the burr hole plug comprising a cover assembly disposed over the burr hole and at least partially extending outwardly from an outer surface of the patient skull,
an electrical stimulation lead configured to extend through the burr hole cover and into the burr hole from a location at least partially external to the patient's skull, and
a pulse generator configured to generate stimulation energy and coupleable with the electrical stimulation lead, the pulse generator configured for implanting into the patient at a location at least partially external to the patient's skull; and
the cranial prosthesis of claim 17, the cranial prosthesis configured to reduce visibility of bulging along the scalp caused by the portion of the electrical stimulation system extending outwardly from the patient's skull when the cranial prosthesis is disposed over a portion of the electrical stimulation system.
